(19) United States
(12) Patent Application Publication
(10) Pub. No.: US 2010/0322984 A1
(43) Pub. Date: Dec. 23, 2010

(54) ORAL CARE COMPOSITION FOR COMBATING DENTINE HYPERSENSITIVITY

(76) Inventors: Gareth David Rees, Surrey (GB); Charles Richard Parkinson, Surrey (GB); Robert Mark Anderson, Shropshire (GB)

Correspondence Address: GlaxoSmithKline;GLOBAL PATENTS -US, UW2220, P. O. BOX 1539, KING OF PRUSSIA, PA, 19406-0939, US

(21) Appl. No.: 12/865,618

(22) PCT Filed: Jan. 29, 2009
(86) PCT No.: PCT/EP2009/050970

§ 371 Date: Jul. 30, 2010

(30) Foreign Application Priority Data
Jan. 31, 2008 (GB) 0801836.8

(51) Int. Cl. A61K 8/02; A61K 8/81; A61Q 11/00

(52) U.S. Cl. 424/401; 424/49; 424/52

(57) ABSTRACT

The present invention describes oral care compositions comprising certain surface-coated inorganic oxides and their use for treating dentine hypersensitivity.

The present invention relates to oral care compositions comprising certain surface-coated inorganic oxides for use in combating dentine hypersensitivity.

Dentine hypersensitivity is a common but painful condition affecting up to 20% of the adult population. The characteristic short, sharp pain of dentine hypersensitivity is triggered by thermal, evaporative, tactile, osmotic or chemical stimuli. The primary origin is generally agreed to result from the exposure of dentine following either loss of the protective enamel layer or via gum recession.

The most pronounced morphological characteristic of human dentine is its tubular structure. Dentine tubules have diameters on the order of several micrometers and connect the pulp to the enamel dentine junction. In a healthy subject, these tubules are filled with fluid. It is postulated that this dental fluid plays an active role in the transmission of pain stimuli across the dentine to the underlying neurons. The most widely-accepted theory, known as the hydrodynamic theory, states that when the dentine tubules become exposed to the environment, external stimuli elicit a displacement of the dentinal fluid, which, in turn, stimulates mechanoreceptors in the pulp. The movement of fluid through the narrow tubules irritates cells in the vicinity of the base of the tubules, including odontoblasts, pulpal neurons, and even subodontonblastic blood vessels. Several researchers have shown that the fluid movements result in the release, from the pulpal nerves, of calcitonin gene-related peptide, which generates a local neurogenic inflammatory condition.

There are two categories of therapy for the treatment of dentine hypersensitivity based upon two modes of action. The first category, nerve-depolarising agents, are pharmaceutical agents such as potassium nitrate, which function by interfering with neural transduction of the pain stimulus.

The second category, known as occluding agents, function by physically blocking the exposed ends of the dentinal tubules, thereby reducing dentinal fluid movement and reducing the irritation associated with the shear stress described by the hydrodynamic theory.

The occlusion approach typically involves treating the tooth with a chemical or physical agent that creates a deposition layer within or over the dentine tubules. This layer mechanically occludes the tubules and prevents or limits fluid movement within the tubule to such an extent that stimulation of the neuron is not achieved. Examples of occlusion actives include among others, calcium salts, oxalate salts, stannous salts, glasses, and varnishes.

U.S. Pat. No. 5,270,031 (Block) relates to water soluble or water swellable polymers with functional groups that are capable of bearing one or more charged groups in an aqueous solution having desensitising properties. Such polymers can be anionic, cationic or amphoteric.

One example of an anionic functional group is the carboxylate group which is found in polymers such as polyacrylic acid, copolymers of acrylic acid and maleic acid, copolymers of methacrylic acid and acrylic acid, and copolymers of alkyl vinyl ethers and maleic acid or anhydride.

U.S. Pat. No. 5,885,551 (Block) relates to a method of treating dentinal hypersensitivity by administering alginic acid or an alginate in an oral care composition.

U.S. Pat. No. 6,096,292 (Block) relates to the use of a superabsorbent acrylic polymer as a desensitising agent.

U.S. Pat. No. 6,241,972 (Block) relates to compositions and their use in treating dentinal hypersensitivity comprising a copolymer having repeated units of a hydrophilic monomer such as a carboxylic acid, a dicarboxylic acid or a dicarboxylic acid anhydride and a hydrophobic monomer consisting of an alpha-olefin having at least eight carbon atoms, full and partially hydrolysed forms thereof and full and partial salts thereof. A preferred desensitising agent is PA-18 which is an alternating copolymer of a 1:1 molar ratio of maleic anhydride and 1-octadecene.

U.S. Pat. No. 5,244,651 (Kao) relates to a method of treating dentinal hypersensitivity with a colloid produced by mixing a salt of a polyvalent metal with a polyol phosphate.

U.S. Pat. No. 5,718,885 (Block) relates to phosphate free oral compositions containing cationically charged colloids for treating tooth hypersensitivity. Examples of charged colloids are those prepared from metal oxides including alumina.

WO 00/59460 (Grace) relates to porous inorganic oxide-based dentifrice additives with particle size in the range 0.05 to 3 microns, for use in tooth sensitivity and remineralisation. Examples of inorganic oxide particles include SiO₂, Al₂O₃, MgO, TiO₂ and ZrO₂.

WO 02/051945 (Henkel) relates to nanoparticulate titanium dioxide with a mean particle diameter ranging from 10 to 1000 nm being coated with a polar organic surface-modifying agent. The particles are described as being suitable as tooth-brightening agents. Preferred surface-modifying agents include substances containing two or more functional groups selected from carboxlic acids, phosphonic acids, amino acids, sulphonic acids and certain silanes. Polymers are not described.

EP 1 630 136A1 (Toto) relates to surface-modified titanium dioxide particles which have a surface chemically modified with a carboxyl-containing hydrophilic polymer having a particle size of 2 to 200 nm, the carboxyl groups in the hydrophilic polymer being bonded to the titanium dioxide through an ester linkage. The surface-modified titanium dioxide is demonstrated to have antimicrobial activity and cytotoxic activity against cancer cells. The present invention is based on the discovery that certain surface-coated inorganic oxides have desensitising properties when measured using a hydraulic conductance model, an established in vitro model for assessing the efficacy of desensitising agents.

Accordingly the present invention provides an oral care composition for combating dentine hypersensitivity comprising inorganic oxide particles surface-coated with an anionic polymer, and an orally acceptable carrier or excipient.

The term anionic polymer refers to a polymer comprising a plurality of anionic functional groups, an example of such a polymer is a polycarboxylate.

Suitably the surface-coated inorganic oxide particles have a mean particle diameter from 0.01 μm to 3 μm, for example from 0.1 to 3 μm, such as from 0.25 μm to 2 μm.

Suitably the inorganic oxide is selected from silica, titanium dioxide or alumina (aluminium oxide, ie Al₂O₃) or a mixture thereof.

Suitably a polycarboxylate is selected from a polyacrylic acid, a copolymer of acrylic acid and maleic acid, a copolymer of methacrylic acid and acrylic acid, or a copolymer of an alkyl vinyl ether and maleic acid or anhydride.

Suitably a polycarboxylate is a polyacrylic acid, for example having a molecular weight of about 1,000 to about 1,000,000, for example from about 10,000 to about 100,000, or from about 20,000 to 50,000. Suitably the polyacrylic acid may be in a neutralised form, for example in the form a sodium or potassium salt.

Suitably a polycarboxylate is a polysaccharide containing carboxy functional groups, for example alginic acid or a derivative thereof such as an alginate.

Suitably a polycarboxylate is a copolymer having repeated units of a hydrophilic monomer selected from a carboxylic acid, a dicarboxylic acid or a dicarboxylic acid anhydride and a hydrophobic monomer consisting of an alpha-olefin having at least eight carbon atoms, full and partially hydrolysed forms thereof and full and partial salts thereof. Such copolymers are described in US 6,241,72 (Block) the contents of which are incorporated herein by reference. An example of such a polycarboxylate is PA-18 which is an alternating copolymer of a 1:1 molar ratio of maleic anhydride and 1-octadecene (referred to as octadecene maleic anhydride copolymer).

Surface-coating may be achieved by covalent bonding of the coating material to the inorganic oxide particle, for example as described in EP 1 630 136A1 (Toto), or by electrostatic means.

Coating may be achieved using either (i) a ‘double coating’ methodology whereby the inorganic oxide particle is first electrostatically coated with a cationic polymer, for example, polyethyleneimine or an amphiphile such as chitosan and then subsequently coated with the desired anionic polymer, or (ii) via a ‘single-layer’ deposition method whereby the anionic polymer is electrostatically deposited onto a positively charged substrate.

Compositions of the present invention suitably comprise from 0.1 to 60.0% w/w surface-coated inorganic oxide, such as from 0.1 to 30.0% w/w of the total composition.

Compositions of the present invention will contain appropriate formulating agents such as abrasives, surfactants, thickening agents, humectants, flavouring agents, sweetening agents, opacifying or colouring agents, pH buffering agents and preservatives, selected from those conventionally used in the oral care composition art for such purposes. Examples of such agents are as described in EP 929287.

The oral compositions of the present invention are typically formulated in the form of toothpastes (including prophy pastes), sprays, mouthwashes, gels, suspensions, varnishes, sealants, coatings, lozenges, chewing gums, tablets, pastilles, instant powders, oral strips and buccal patches.

Oral care actives may also be included in the compositions of the present invention, including additional desensitising agents. Examples of desensitising agents include tubule blocking agents or nerve desensitising agents and mixtures thereof, for example as described in WO 02/15809. Suitable desensitising agents include a strontium salt such as strontium chloride, strontium acetate or strontium nitrate or a potassium salt such as potassium citrate, potassium chloride, potassium bicarbonate, potassium gluconate and especially potassium nitrate.

Compositions of the present invention may further comprise a source of soluble fluoride ions such as those provided by sodium fluoride, sodium monofluorophosphate, tin (II) fluoride or an amine fluoride in an amount to provide from 25 to 3500 ppm fluoride, such as from 100 to 1500 ppm.

The compositions according to the present invention may be prepared by admixing the ingredients in the appropriate relative amount in any order that is convenient.

The present invention also provides a method of combating dentine hypersensitivity which comprises applying an effective amount of a composition as herein before defined to an individual in need thereof.

The present invention further provides use of an oral care composition as herein before defined in the manufacture of a medicament for treating dentine hypersensitivity.

The invention is further illustrated by the following Examples:

### Surface-Coating Methodology

Method (i)—The Double Coating Method was utilised to coat silica. Polyethyleneimine (PEI) was selected as the primer.

Method (ii)—The Single Layer Method was utilised to coat titanium oxide and aluminium oxide. Method (ii) involves adjusting the pH of the inorganic oxide particle environment such that it attains a positive surface charge.

Both coating techniques detailed above utilised the same general coating methodology and, unless specified, the coating was carried out by addition of a 10 w/w % slurry of particles in water to a 4-5 w/w % solution of polymer, and left to stir for at least 16 hours. The resulting suspension was then purified by cross-flow filtration through a 0.1 m Minikros module, washing with a total of ten volumes of reverse osmosis water with respect to the amount of initial solution and then concentrated to the desired level, in the range of 3-10 w/w %.

Variations from this general method were necessary for certain polymer systems in which complete purification could not be achieved due to the slow filtration of suspension or repeated blockage of the membranes, as a consequence of the aggregated solution size of the polymer.

### In-vitro Testing Methodology

The polymer coated inorganic oxide particle composite technology was evaluated in the hydraulic conductance (Hc) model using a protocol designed to explore the initial occlusion capability of new occlusional actives. The Hc model has been used extensively to assess the potential of desensitising agents to occlude dentinal tubules.

Briefly, dentine permeability as a function of hydraulic conductance is measured for each specimen (dentine disk) at baseline and after treatment. Thereby, each disk serves as its own control: Prior to treatment the hydraulic conductance of each specimen is measured following application of a salivary pellicle. This value is set to represent 100% permeability, and is termed the baseline permeability of the disk. After application of the test active the hydraulic conductance is re-measured (over 5 minute time period). This value is used to calculate a percentage permeability reduction for the particular test active. For optimized samples the test active was reapplied up to four times. Following treatment the disks are subjected to simulated oral challenges. In order, these consist of (i) a 30 sec. brush while rinsed with 0.17% w/w KCl, and (ii) the application of a 10 sec. purge pressure (12 p.s.i. on the pulpal side of the disk). Up to three dentine disks were used in each treatment group.

### Results

Table 1, below details the results of Hc testing on five polymer-particle composites and an uncoated control (Aluminium oxide).

| | | | | Hc data (% reduction in fluid flow w.r.t | | | |
|---|---|---|---|---|---|---|---|
| | | | | saliva baseline). SD in parentheses. | | | |
| | | | Loading | Initial | % red. in | % red. in Hc on | |
| | | | (solids) | reduction | Hc after 4 | (a) brushing, | |
| Coating | Primer | Substrate | (% w/v) | in Hc | applications | (b) purge. | |
| Polyacrylic | None | Titanium | 10.0 | 19.8 (2.5) | 39.6 (2.3) | (a) | 27.0 |
| acid (neutralized) | | oxide | | | | (b) | 10.0 |
| Polyacrylic | None | Aluminium | 9.5 | 20.2 (2.0) | 23.7 (0.8) | (a) | 4.0 |
| acid (neutralized) | | oxide | | | | (b) | 0.0 |
| Polyacrylic | Polyethylenimine | Silica | 5.0 | 25.0 (5.3) | Not carried out | Not carried out | |
| acid (neutralized) | | | | | | | |
| PA-18 | None | Aluminium | 5.0 | 15.3 (4.7) | 28.7 (0.5) | (a) | 13.0 |
| | | oxide | | | | (b) | 4.0 |
| Alginate | Polyethylenimine | Silica | 10.0 | 26.0 (7.7) | Not carried out. | Not carried out. | |
| None | None | Aluminium | 10.0 | 3.2 (2.6) | −1.9 (5.0) | (a) | −0.7 |
| | | oxide | | | | (b) | −4.8 |

Graph 1 shows results observed for polyacrylic acid (PAA)-coated TiO₂ with respect to another control (untreated TiO₂) as a function of treatment stage.

Graph 2 shows a montage of PAA-coated titanium dioxide, PAA-coated aluminium oxide, PA-18 coated aluminium oxide and for control uncoated aluminium oxide.

1. An oral care composition for combating dentine hypersensitivity comprising inorganic oxide particles surface-coated with an anionic polymer, and an orally acceptable carrier or excipient.

2. A composition according to claim 1 wherein the anionic polymer is a polycarboxylate.

3. A composition according to claim 1 wherein the surface-coated inorganic oxide particles have a mean particle size from 0.01 μm to 3.0 μm.

4. A composition according to claim 1 wherein the inorganic oxide is selected from silica, titanium dioxide, or alumina or a mixture thereof.

5. A composition according to claim 1 wherein the polycarboxylate is a polyacrylic acid, a copolymer of acrylic acid and maleic acid, a copolymer of methacrylic acid and acrylic acid, or a copolymer of an alkyl vinyl ether and maleic acid or anhydride.

6. A composition according to claim 1 wherein the polycarboxylate is a polyacrylic acid.

7. A composition according to claim 1 wherein the polycarboxylate is a polysaccharide containing carboxy functional groups.

8. A composition according to claim 7 wherein the polycarboxylate is an alginic acid or derivative thereof.

9. A composition claim 1 wherein the polycarboxylate is a copolymer having repeated units of a hydrophilic monomer selected from a carboxylic acid, a dicarboxylic acid or a dicarboxylic acid anhydride and a hydrophobic monomer consisting of an alpha-olefin having at least eight carbon atoms, full and partially hydrolysed forms thereof and full and partial salts thereof.

10. A composition according to claim 9 wherein the polycarboxylate is an octadecene maleic anhydride copolymer.

11. A composition according claim 1 wherein the surface-coated inorganic oxide particles are present in an amount of 0.1 to 60% w/w of the total composition.

12. A composition according to claim 1 further comprising an additional desensitising agent.

13. A composition according to claim 1 further comprising a source of soluble fluoride ions.

14. A method of combating dentine hypersensitivity which comprises applying an effective amount of a composition as defined in claim 1 to an individual in need thereof.
